(19) United States
(12) Patent Application Publication
(10) Pub. No.: US 2018/0263478 A1
(43) Pub. Date: Sep. 20, 2018

(54) ENDOSCOPE TUBING

(71) Applicant: MEDICAL INTUBATION TECHNOLOGY CORPORATION, Taoyuan City (TW)

(72) Inventors: Che-Li CHANG, TAOYUAN CITY (TW); Ming-Yuan LIN, TAOYUAN CITY (TW)

(21) Appl. No.: 15/922,072

(22) Filed: Mar. 15, 2018

(30) Foreign Application Priority Data
Mar. 17, 2017 (TW) 106108964

(51) Int. Cl. A61B 1/05; G02B 23/24; A61B 1/00

(52) U.S. Cl. CPC A61B 1/05; G02B 23/26; A61B 1/00137; G02B 23/2476

(57) ABSTRACT

An endoscope tubing includes: a picture-taking component defined with a front end and a rear end and having an image-capturing lens disposed at the front end to capture images forward, a light source and an outer wall, with the light source disposed at a rim of the image-capturing lens to illuminate, and the outer wall being circular and protruding toward the front of the picture-taking component to form a protruding dam; a spring tube with an end connected to the rear end of the picture-taking component and another end connected to a predetermined device; a tensile cable with a portion thereof connected to the picture-taking component and another portion thereof passing through the spring tube to connect to the predetermined device; and a wire with an end connected to the picture-taking component and another end passing through the spring tube to connect to the predetermined device.

### BACKGROUND OF THE INVENTION

### 1. Technical Field

The present invention relates to endoscopes and, more particularly, to an endoscope tubing.

### 2. Description of Related Art

Taiwan patent M486911 discloses an endoscope which essentially comprises a host adapted to be handheld. The host has a screen and required function buttons. A signal connection receptacle is disposed at the front end of the host. An extensible rod of the endoscope is inserted into and connected to the signal connection receptacle. A signal line is disposed in the extensible rod and connected to a lens disposed at the front end of the extensible rod. LED lamps fill the space of a gap between the rim of the lens and the wall of the extensible rod.

The lens is always in the forefront of the endoscope in operation and thus has to be maneuvered around obstacles in order to move forward. As a result, the lens confronted with the obstacles is likely to get damaged. Although Taiwan patent M486911 discloses an endoscope with an extensible rod filled with a plurality of LEDs for supplementing illumination while the lens is taking pictures and capturing images with a view to enhancing the quality of the images captured, Taiwan patent M486911 does not disclose any means of allowing the lens to turn efficiently or protecting the lens. In view of this, there is still room for improvement in allowing lenses of endoscopes to turn efficiently and protecting the lenses.

### BRIEF SUMMARY OF THE INVENTION

It is an objective of the present invention to provide an endoscope tubing that allows a lens to turn efficiently and protect the lens better.

In order to achieve the above and other objectives, the present invention provides an endoscope tubing, comprising: a picture taking component not only having a casing but also having an image-capturing lens and at least a light source which are disposed at the casing, the image-capturing lens being disposed at a front end of the casing to capture images forward, the at least a light source being disposed at a rim of the image-capturing lens to illuminate forward, wherein an outer wall of the casing is circular and is of a diameter which increases gradually in a direction from a front end thereof to a rear end thereof and then decreases gradually in a direction from the front end to the rear end, and the casing protrudes forward to form a protruding dam which reaches a more forward position than the image-capturing lens and surrounds the image-capturing lens and the light source; a spring tube with an end connected to a rear end of the picture taking component and another end connected to a predetermined device; a tensile cable with a portion thereof connected to the picture taking component and another portion thereof passing through the spring tube connected to the predetermined device; and at least a wire with an end connected to the picture taking component and another end passing through the spring tube to electrically connect to the predetermined device.

Therefore, an endoscope tubing provided according to the present invention is characterized by advantageous technical features as follows: the outer wall of the casing of the picture taking component is circular, and the casing protrudes forward to form the protruding dam, so as to allow the picture taking component to turn efficiently and allow the image-capturing lens to be better protected.

### BRIEF DESCRIPTION OF THE SEVERAL VIEWS OF THE DRAWINGS

FIG. 1 is a perspective view of an endoscope tubing according to the first preferred embodiment of the present invention;

FIG. 2 is a partial exploded view of the endoscope tubing according to the first preferred embodiment of the present invention;

FIG. 3 is a partial cross-sectional view of the endoscope tubing according to the first preferred embodiment of the present invention;

FIG. 4 is a schematic view of how to operate the endoscope tubing according to the first preferred embodiment of the present invention;

FIG. 5 is a perspective view of the endoscope tubing according to the second preferred embodiment of the present invention; and

FIG. 6 is a partial cross-sectional view of the endoscope tubing according to the second preferred embodiment of the present invention.

### DETAILED DESCRIPTION OF THE INVENTION

Technical features of the present invention are illustrated by the first preferred embodiment and the accompanying drawings and described below.

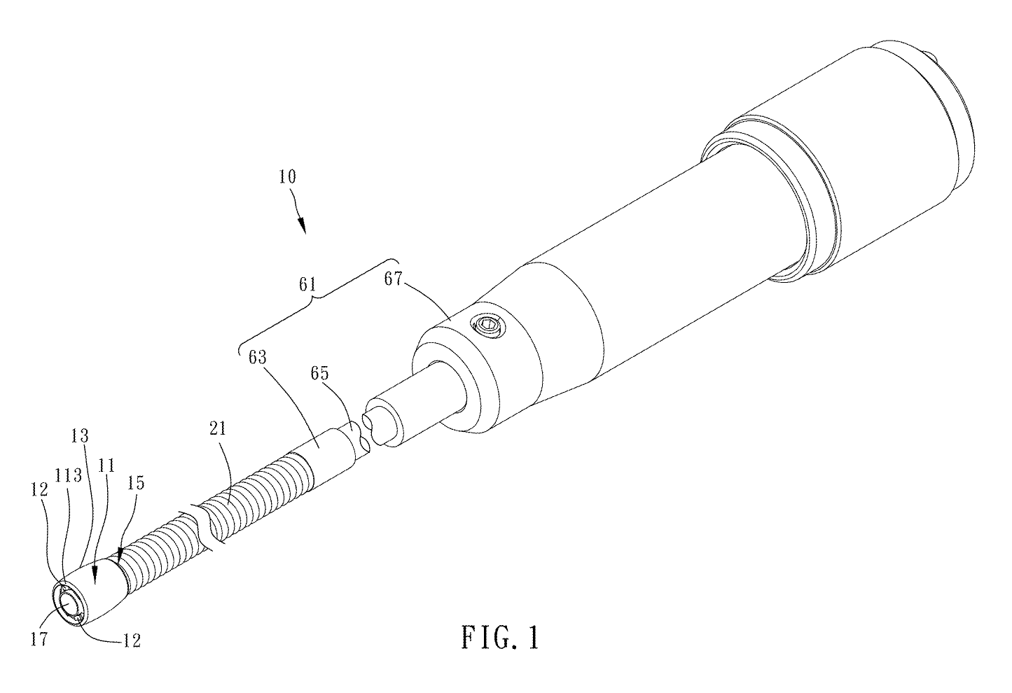

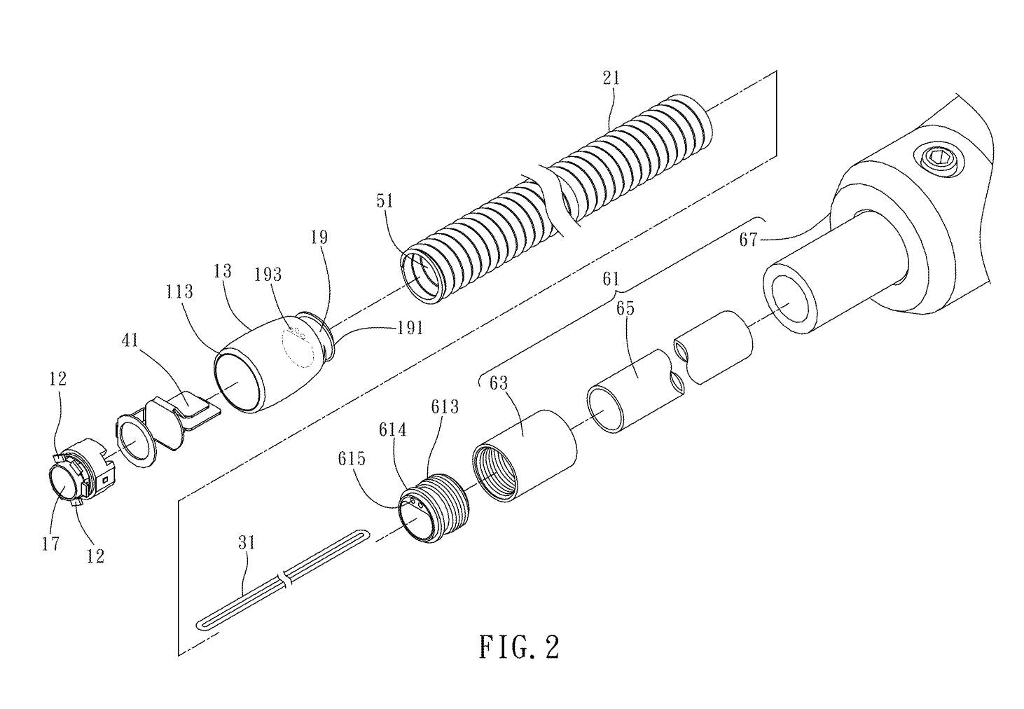

Referring to FIG. 1 through FIG. 3, an endoscope tubing 10 provided in the first preferred embodiment of the present invention essentially comprises a picture taking component 11, a spring tube 21, a tensile cable 31 and at least a wire 41 (in this preferred embodiment, the at least a wire 41 is provided in a plural number for exemplary purpose but shown as a single wire because the plurality of wires 41 is attributed to the prior art.)

The picture taking component 11 not only has a casing 13 but also has an image-capturing lens 17 and at least a light source 12 which are disposed at the casing 13. The image-capturing lens 17 is disposed at the front end of the casing 13 to capture images forward. The at least a light source 12 is disposed at the rim of the image-capturing lens 17 to illuminate forward. The outer wall of the casing 13 is circular and is of a diameter which increases gradually in the direction from a front end thereof to a rear end thereof and then decreases gradually in the direction from the front end to the rear end. The casing 13 protrudes forward to form a protruding dam 113. The protruding dam 113 reaches a more forward position than the image-capturing lens 17 and surrounds the image-capturing lens 17 and the light source 12. The casing 13 is circular; hence, while it is moving, the picture taking component 11 of the endoscope tubing 10 not only rotates efficiently but is also less likely to jam. From a structural perspective, the fact that the protruding dam 113 reaches a more forward position than the image-capturing lens 17 and surrounds the image-capturing lens 17 and the light source 12 enables the protruding dam 113 to confront with any obstacle and thus prevent the image-capturing lens 17 from colliding with or brushing over the obstacle. However, the length by which the protruding dam 113 protrudes toward the front of the image-capturing lens 17 must not interfere with the image-capturing function of the image-capturing lens 17 with a view to preventing deterioration of the quality of the images captured.

One end of the spring tube 21 connects to a rear end 15 of the picture taking component 11. The other end of the spring tube 21 connects to a predetermined device 61. The spring tube 21 is a tight spring. Coils of the spring tube 21 are packed tightly together to have no gaps between them when the spring tube 21 is not bent. The spring tube 21 enables the endoscope tubing 10 to have both rigidity and flexibility advantageously. Owing to its rigidity, the endoscope tubing 10 is resistant to abrasion and thus has a long service life.

A portion of the tensile cable 31 is connected to the picture taking component 11. The other portion of the tensile cable 31 passes through the spring tube 21 to connect to the predetermined device 61.

One end of each of the plurality of wires 41 connects to the picture taking component 11. The other end of each of the plurality of wires 41 passes through the spring tube 21 connected to the predetermined device 61.

In this preferred embodiment, the predetermined device 61 comprises a sleeve 63 and a flexible tube 65. The flexible tube 65 has one end connected to the sleeve 63 and the other end connected to an operating handle 67. The flexible tube 65 has therein the plurality of wires 41 and thereby is electrically connected to the sleeve 63 and the operating handle 67. The rear end 15 of the picture taking component 11 extends rearward to form a connection portion 19. The connection portion 19 has a first thread 191. One end of the sleeve 63 has a fixing element 613. A portion of the fixing element 613 is disposed in the sleeve 63. The other portion of the fixing element 613 is exposed from the sleeve 63. The fixing element 613 has a second thread 614. The second thread 614 is disposed on the outer side of a portion of the fixing element 613, and the portion of the fixing element 613 is exposed from the sleeve 63. Two ends of the spring tube 21 are connected to the first thread 191 and the second thread 614, respectively, by a screwing means.

Although the aforesaid technical means of connecting the spring tube 21 to the connection portion 19 and the fixing element 613 is exemplified by a screwing means, it can also be a gluing means or a welding means. Therefore, the present invention is not restrictive of the means of connecting the spring tube 21 to the connection portion 19 and the fixing element 613.

The inner wall of the sleeve 63 further has an inner dam 631. The fixing element 613 abuts against the inner dam 631 from one side thereof. The fixing element 613 and the sleeve 63 are fixed together by a means of screwing. The flexible tube 65 abuts against the inner dam 631 from the other side thereof. The outer wall of the flexible tube 65 is fixedly disposed on the inner wall of the sleeve 63. The inner dam 631 of the sleeve 63 separates the flexible tube 65 from the fixing element 613.

The connection portion 19 further has two first through holes 193. The two first through holes 193 penetrate the connection portion 19. The fixing element 613 further has two second through holes 615. The two second through holes 615 are penetratingly disposed in the fixing element 613 in the axial direction of the sleeve 63. The tensile cable 31 is ring-shaped. The ring-shaped tensile cable 31 encirclingly connects to the two first through holes 193 and the two second through holes 615.

The structure of the endoscope tubing 10 in the first preferred embodiment of the present invention is described above. The operation of the endoscope tubing 10 in the first preferred embodiment of the present invention is described below.

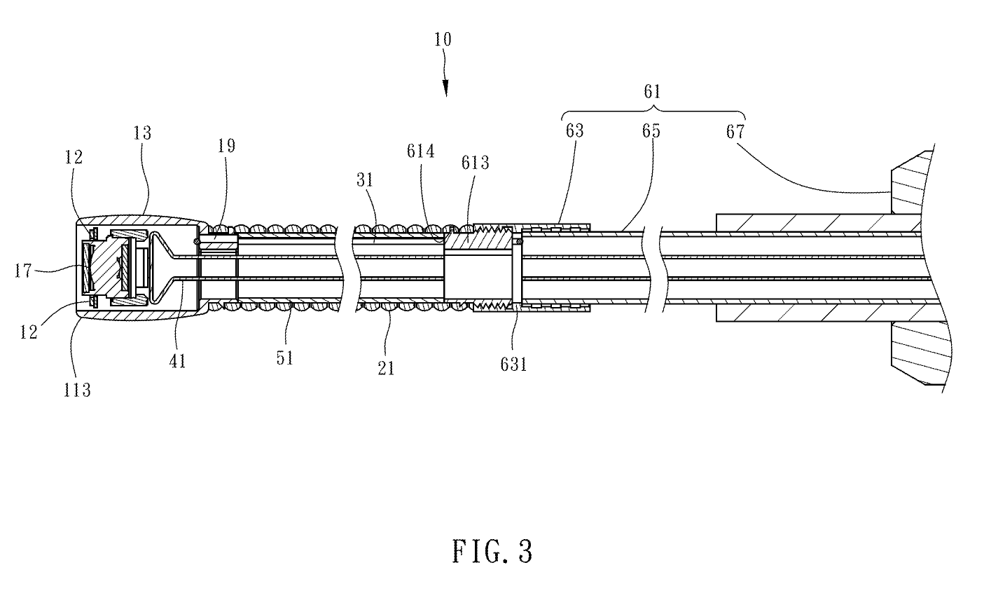

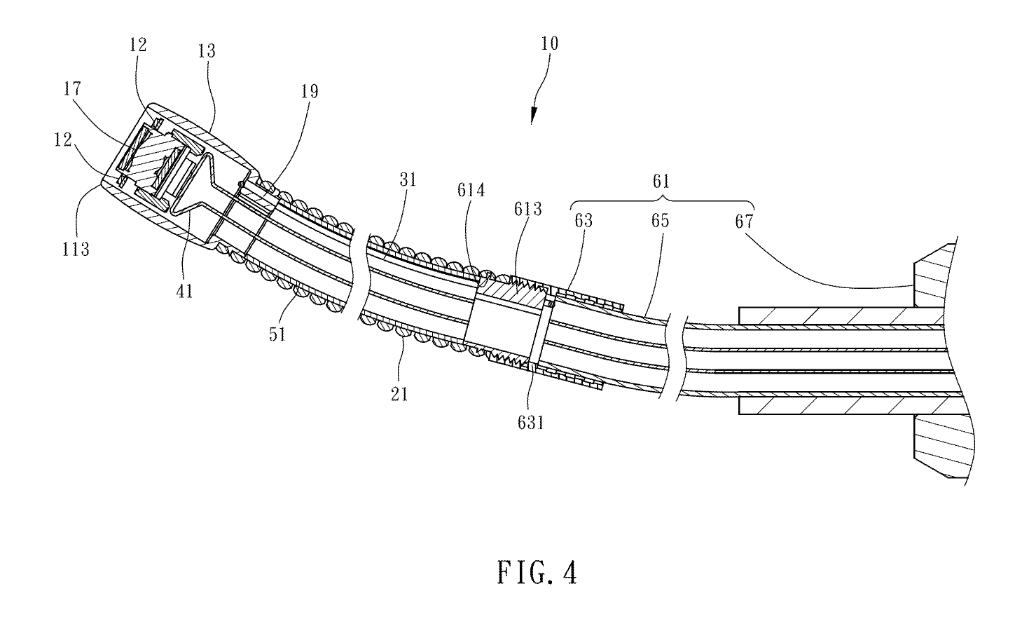

Referring to FIG. 3 and FIG. 4, operation of the endoscope tubing 10 begins with inserting the picture taking component 11 into an object to be examined and capturing images of the object from inside by a user. As soon as the picture taking component 11 is confronted with a bend or an obstacle in the course of its advance, the flexibility of the spring tube 21 enables the picture taking component 11 to follow the bend or turn around the obstacle. The outer wall of the casing 13 of the picture taking component 11 is circular such that the picture taking component 11 turns easily (as shown in FIG. 4). The casing 13 protrudes forward to form the protruding dam 113 whereby the image-capturing lens 17 is better protected. Owing to the rigidity of the spring tube 21, the endoscope tubing 10 features enhanced resistance to abrasion to not only provide enhanced protection for the inside of the spring tube 21 but also extend the service life of the endoscope tubing 10.

Upon completion of the image-capturing process performed with the endoscope tubing 10, the user take the picture taking component 11 out of the object slowly. To terminate the jam whenever the picture taking component 11 has been jammed by an obstacle, the user may push and pull the endoscope tubing 10 slightly and repeatedly or may shake the endoscope tubing 10 from outside the object (to cause the endoscope tubing 10 to shake inside object.) Since the casing 13 of the picture taking component 11 is circular, the aforesaid jam which occurs to the picture taking component 11 of the endoscope tubing 10 can be terminated more easily than the jam which occurs to a conventional endoscope tubing. Furthermore, the aforesaid user's persistent pushing, pulling and shaking of the endoscope tubing 10 is likely to cause the spring tube 21 to sever or deform in the user's ignorance while being unaware of what is going on inside the object, because the inside of the object is invisible to the user. The severance or deformation of the spring tube 21 is likely to cause damage to electronic components inside the spring tube 21. Fortunately, tensile strength of the tensile cable 31 is high enough to prevent the spring tube 21 from being severed or deformed under a pulling force.

Hence, the objective of the present invention is achieved, as the endoscope tubing 10 is characterized by advantageous technical features as follows: the outer wall of the casing 13 of the picture taking component 11 is circular, and the casing 13 protrudes forward to form the protruding dam 113, so as to allow the picture taking component 11 to turn efficiently and allow the image-capturing lens 17 to be better protected.

In addition, the endoscope tubing 10 further has an inner flexible tube 51, and the inner flexible tube 51 is disposed in the spring tube 21. Although the endoscope tubing 10 features enhanced resistance to abrasion because of the rigidity of the spring tube 21, other electronic components (not shown) are usually electrically connected to the spring tube 21 from inside. The inner flexible tube 51 not only prevents the spring tube 21 from brushing against the electronic components in the spring tube 21, but also reduces accumulation of dust in the spring tube 21 while the endoscope tubing 10 is in use. However, the inner flexible tube 51 will be dispensable, if the operating environment is free of dust. Hence, the inner flexible tube 51 is not necessarily a required element for achieving the objective of the endoscope tubing 10.

The endoscope tubing 10 further has a plastic component (not shown, because a depiction of the plastic component in operation is difficult.) The plastic component is disposed at the junction of the spring tube 21 and the first thread 191 and at the junction of the spring tube 21 and the second thread 614 to augment the stability of the connection of the spring tube 21 and the first thread 191 and the stability of the connection of the spring tube 21 and the second thread 614, thereby preventing detachment of either or both ends of the spring tube 21 from the first thread 191 and the second thread 614 under a pulling force. However, the plastic component will be dispensable, if its presence in the operating environment is inappropriate or excessive pulling seldom occurs in the operating environment.

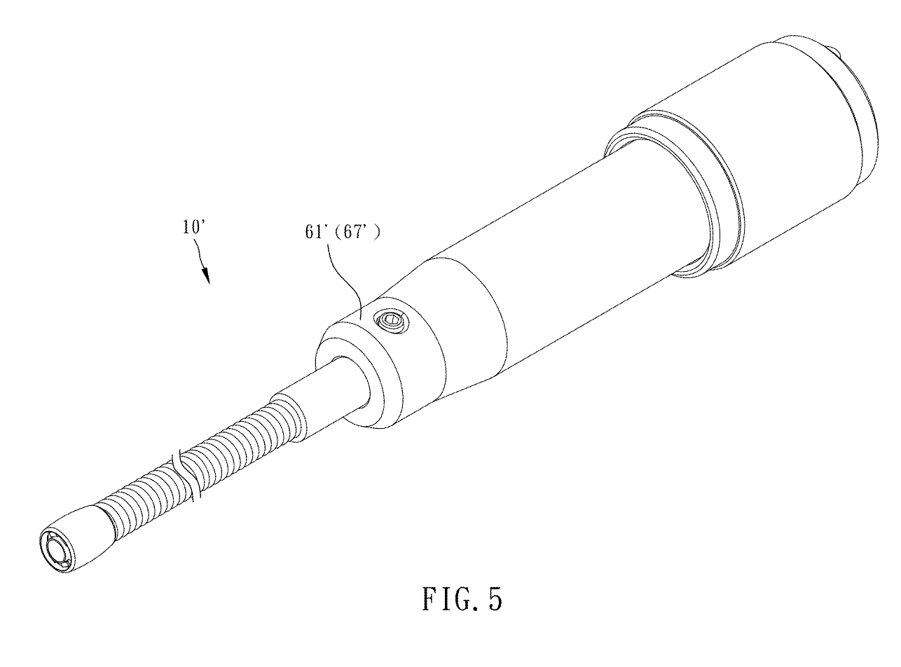

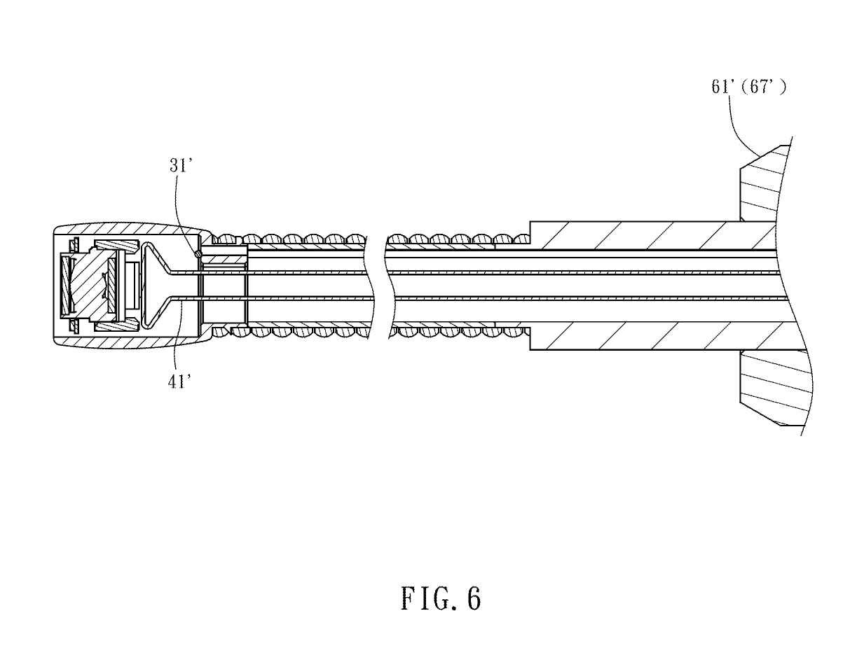

Referring to FIG. 5 and FIG. 6, an endoscope tubing 10′ provided in the second preferred embodiment of the present invention is substantially the same as the endoscope tubing 10 provided in the first preferred embodiment of the present invention, except for the distinguishing technical features described below.

The predetermined device 61′ comprises an operating handle 67′, exclusive of the sleeve 63 and the flexible tube 65, as shown in FIG. 1. The wires 41′ are electrically connected to the operating handle 67′. The tensile cable 31′ connects to the operating handle 67′. Other structural features of the second preferred embodiment and advantages achieved by the other structural features are substantially the same as those of the first preferred embodiment and thus are not described herein again for the sake of brevity.

What is claimed is:

1. An endoscope tubing, comprising:
a picture taking component not only having a casing but also having an image-capturing lens and at least a light source which are disposed at the casing, the image-capturing lens being disposed at a front end of the casing to capture images forward, the at least a light source being disposed at a rim of the image-capturing lens to illuminate forward, wherein an outer wall of the casing is circular and is of a diameter which increases gradually in a direction from a front end thereof to a rear end thereof and then decreases gradually in a direction from the front end to the rear end, and the casing protrudes forward to form a protruding dam which reaches a more forward position than the image-capturing lens and surrounds the image-capturing lens and the light source;
a spring tube with an end connected to a rear end of the picture taking component and another end connected to a predetermined device;
a tensile cable with a portion thereof connected to the picture taking component and another portion thereof passing through the spring tube connected to the predetermined device; and
at least a wire with an end connected to the picture taking component and another end passing through the spring tube to electrically connect to the predetermined device.

2. The endoscope tubing of claim 1, wherein the predetermined device comprises a sleeve and a flexible tube, the flexible tube having an end connected to the sleeve and another end connected to an operating handle, the flexible tube having therein a plurality of wires and thereby being electrically connected to the sleeve and the operating handle, and the rear end of the picture taking component extends rearward to form a connection portion, the connection portion having a first thread, with a fixing element disposed at an end of the sleeve, the fixing element having a portion thereof disposed in the sleeve and another portion thereof exposed from the sleeve, the fixing element having a second thread, wherein the second thread is disposed on an outer side of a portion of the fixing element, and the portion of the fixing element is exposed from the sleeve, wherein two ends of the spring tube connect to the first thread and the second thread, respectively, by screwing.

3. The endoscope tubing of claim 2, wherein an inner wall of the sleeve further has an inner dam, with the fixing element abutting against the inner dam from a side thereof, allowing the fixing element and the sleeve to be fixed together by a means of screwing, and the flexible tube abuts against the inner dam from another side thereof, allowing an outer wall of the flexible tube to be fixedly disposed on the inner wall of the sleeve.

4. The endoscope tubing of claim 2, wherein the connection portion further has two first through holes penetrating the connection portion, and the fixing element further has two second through holes penetratingly disposed in the fixing element in an axial direction of the sleeve, with the tensile cable being ring-shaped and encirclingly connecting to the two first through holes and the two second through holes.

5. The endoscope tubing of claim 2, further comprising a plastic component disposed at a junction of the spring tube and the first thread and at a junction of the spring tube and the second thread.

6. The endoscope tubing of claim 1, wherein the spring tube is a tight spring, and coils are packed tightly together to have no gaps between them when the spring tube is not bent.

7. The endoscope tubing of claim 1, further comprising an inner flexible tube disposed in the spring tube.

8. The endoscope tubing of claim 1, wherein the predetermined device comprises an operating handle connecting to the at least a wire, and the tensile cable connects to the operating handle.
